(19) United States
(12) Patent Application Publication
(10) Pub. No.: US 2026/0102276 A1
(43) Pub. Date: Apr. 16, 2026

(54) Mandibular Advancement Device (MAD) having adjustment features

(71) Applicant: Sentient Reality Dental, LLC, Dallas, TX (US)

(72) Inventors: Mark S. Quadling, Plano, TX (US); Henley S. Quadling, Dallas, TX (US)

(21) Appl. No.: 19/355,632

(22) Filed: Oct. 10, 2025

Related U.S. Application Data
(60) Provisional application No. 63/705,902, filed on Oct. 10, 2024.

(51) Int. Cl. A61F 5/56; B33Y 80/00

(52) U.S. Cl. CPC A61F 5/566; B33Y 80/00

(57) ABSTRACT

A dental article of manufacture comprising a first appliance, and a second appliance. The first appliance fits dentition of a patient’s lower dental arch, and the second appliance fits dentition of the patient’s upper dental arch. The first appliance has a first feature positioned at its midline and located at a given distal offset with respect to an incisal edge when fitted on the lower dental arch. Preferably, the second appliance has a second cooperating feature positioned at its midline and includes a closed cavity having a front-facing opening. The second feature is designed to mate with the first feature. To this end, the second feature is positioned to removably receive the first feature only through the facing opening. In use, the second feature retains the first feature in the closed cavity against backwards and up or down movement when the second appliance is fitted on the upper dental arch.

### BACKGROUND

A Mandibular Advancement Device (“MAD”) is a well-known solution for moving a user’s lower jaw forward relative to the upper jaw. Typically, the device comprises two portions that are attached in some way, either rigidly or in a flexible manner. The first portion attaches to the dentition of the upper jaw and the second portion attaches to the dentition of the lower jaw. In use, the connection between the two portions causes the lower jaw to move forward with respect to the upper jaw by some predetermined distance. Typically, this movement is recommended for patients with obstructive sleep apnea or in milder cases, snoring, and the device works by reducing the ability of the airway to collapse when the patient is sleeping on his or her back.

There are many known appliances of this type. Generally, such appliances can be placed into two main categories. In the first category, the connection between the upper and lower portions is rigid, and in some cases a mechanical linkage such as a screw or rachet type interface is used to advance the lower jaw with respect to the upper jaw. This technique has the benefit of not requiring flexible straps or attachments, which can break and/or need to be replaced periodically due to other wear, but it also has the disadvantage of forcing the upper and lower jaws to be in a rigid position, which can exacerbate clenching issues. In the second category, the upper and lower portions are connected with one or more elastic type straps or bands. This flexible attachment causes the lower portion to move forward with respect to the upper portion (and thereby also causing the lower jaw to move forward with respect to the upper jaw), but it also has the added benefit of allowing some relative motion between the upper and lower jaws.

### SUMMARY

According to this disclosure, a mandibular advancement device is provided that has the benefit of both techniques. In use, the device moves the lower jaw forward with respect to the upper jaw without rigidly locking both in place, while also not requiring the use of elastic bands or straps. Furthermore, the device has the further additional benefits that is entirely printed in 3D (typically in a 3D printer), without the need of any other additional pieces such as elastic bands or straps or any other non-3D printed piece. Besides being easier and cheaper to manufacture, a design of the device lends itself to being delivered digitally, for example, to a dental office, where it can then be 3D printed on location.

According to an aspect of this disclosure, a dental article of manufacture comprises a first appliance, and a second appliance. The first appliance is configured to fit dentition of a patient’s lower dental arch and has a first midline, and the second appliance is configured to fit dentition of the patient’s upper dental arch and has a second midline. The first appliance includes a first feature positioned at the first midline, with the first feature being located at a given distal offset with respect to an incisal edge of the patient’s lower dental arch when the first appliance is fitted on the patient’s lower dental arch. The second appliance comprises a second feature positioned at the second midline and includes a closed cavity having a front-facing opening. The second feature is designed to mate with the first feature. To this end, the second feature is positioned without the given distal offset and configured to removably receive the first feature only through the front-facing opening. In use, the second feature retains the first feature in the closed cavity against backwards and up or down movement when the second appliance is fitted on the patient’s upper dental arch. The first feature and second feature couple to each other at a single-point-of-contact and without requiring additional connectors.

The subject device also has a functional dental benefit by having only a single point of contact near the front of the mouth, and it allows the relative angle and position of the lower appliance (and consequently the lower jaw) to move as required. In use, the device prevents the clenching of the jaw muscles that can cause pain, fatigue or headaches.

### BRIEF DESCRIPTION OF DRAWINGS

FIG. 1 shows the typical position of an individual’s upper and lower dental arches in a relaxed neutral position such as while the individual is sleeping;

FIG. 2 shows an example of a base appliance shape according to this disclosure, where the interior features thereof allow the appliance to be placed on the individual’s dental arch, and with the outside surface depicted as flattened;

FIG. 3 shows the additional feature added to the lower appliance in accordance with a first embodiment of a mandibular advancement device of this disclosure;

FIG. 4 shows an example where the additional feature has a larger offset in a distal direction, corresponding to a larger required forward movement of the lower jaw with respect to the upper jaw;

FIG. 5 depicts a feature added to a front top surface of the upper appliance model in accordance with the first embodiment of the mandibular advancement device of this disclosure;

FIG. 6 illustrates a detailed view of a configuration of the lower feature, also referred to as a lower touch feature;

FIG. 7 demonstrates several views showing the positioning of the lower touch feature inside the upper feature;

FIG. 8 shows the same configuration as in FIG. 7, but with the dental arches also being depicted and showing how the lower feature offset forces incisal edge-to-edge positioning;

FIG. 9 demonstrates the situation where the more distal offset of the lower feature as shown in FIG. 4 is used;

FIG. 10 shows the effect on the lower jaw relative positioning to the upper jaw when the MAD in FIG. 9 is employed;

FIG. 11 below depicts the lower and upper additional features, with the lower feature being received in the upper feature;

FIG. 12 is a bottom view of the coupled features depicting how the lower feature is not fixed but rather has a small envelope of movement; and

FIGS. 13A through 13D depict an alternative embodiment of the device wherein the above-described features are reversed.

### DETAILED DESCRIPTION

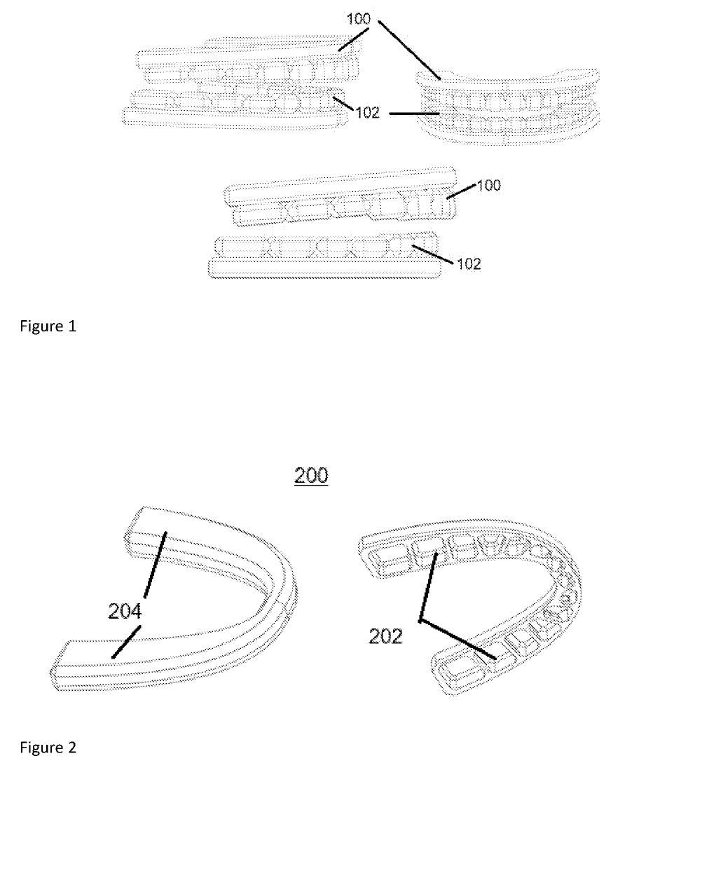

FIG. 1 shows several views (perspective, and front) of the typical position of an individual’s upper and lower dental arches 100 and 102 in a relaxed neutral position while the individual is sleeping. As noted, the purpose of the mandibular advancement device of this disclosure is to move the lower jaw forward relative to the upper jaw, and this is done by adjusting the relative position of the dental arches (the dentition), which in turn are attached to the jaw bones; in this way, the beneficial relative motion is performed.

In the subject approach, the lower and upper devices are designed by first obtaining a digital model of the individual’s dental arches. These may be obtained directly through the use of an intra-oral digitizer or a CBCT Cone Beam scanning system, or indirectly through the means of dental impressions which are then in turn scanned with a 3D digitizer, or first used to generate a physical model which is then scanned with a 3D digitizer. Appliances for the individual’s lower and upper arches are then designed to fully cover the dentition. Preferably, the dentition is fully covered to obtain the maximum retention force. In addition, fully covering the dentition has other dental benefits, such as preventing the super eruption of any teeth which could happen if they were not covered.

To this end, a first step is to calculate a solid model described by a closed 3D surface for both the upper and lower surfaces. For example, if an area on the scanned arch is selected, where that area represents the inside surface of a device to be placed on that arch, then that selected area is used to calculate the inside surface of the appliance. If a zero gap on the inside is desired, then that selected area is used as is for the inside surface, otherwise, an offset surface is calculated, where the offset is determined by the desired internal gap. Likewise, an outer surface is calculated in a similar manner by taking an offset from that inside surface by an amount equal to the desired thickness of the appliance. This method is a well understood and standard manner to calculate a 3D enclosed surface representing a shell-like appliance that fits on top of the arch. The outer surface is then further modified as required, for example, through smoothing or other means to flatten the surface features if necessary.

FIG. 2 shows several perspective views of a base appliance shape 200 where the interior features 202 allow the appliance 200 to be placed on the dental arch, and the outside surface 204 is shown as being flattened. These shapes are for illustration purposes, as typically the appliances themselves are more organically shaped due to the actual dentition of the individual, although the basic principles are the same.

According to this disclosure, a second step in the design process adds an additional feature to the lower appliance. This is sometimes referred to herein as a lower feature, or lower structural element.

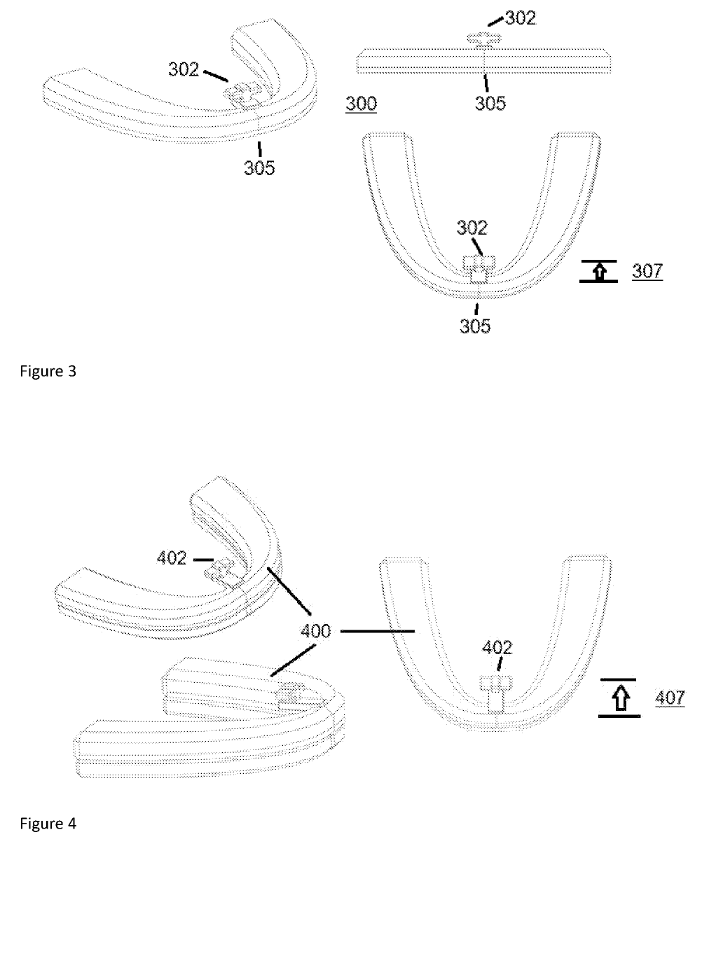

FIG. 3 shows several views (perspective, front, and plan) of the additional feature 302 added to the lower appliance 300. In the depicted embodiment, the feature 303 is positioned on an approximate midline 305 of the dental arch, and at some offset distance 307 (as best seen in the plan view) back from an incisal edge of the underlying dental arch. Note that the midline 305 is referenced here with respect to the appliance for convenience of illustration. This offset distance 307 is determined, e.g., by the desired distance to move the lower jaw forward with respect to the upper jaw. As will become apparent below, as this lower feature 302 is moved distally (i.e. towards the back of the mouth), the effect is to force the lower jaw forward.

FIG. 4 shows a second example where the additional feature 402 of the lower appliance 400 has a larger offset 407 (as compared to offset 307 in FIG. 3) in the distal direction, corresponding to a larger required forward movement of the lower jaw with respect to the upper jaw.

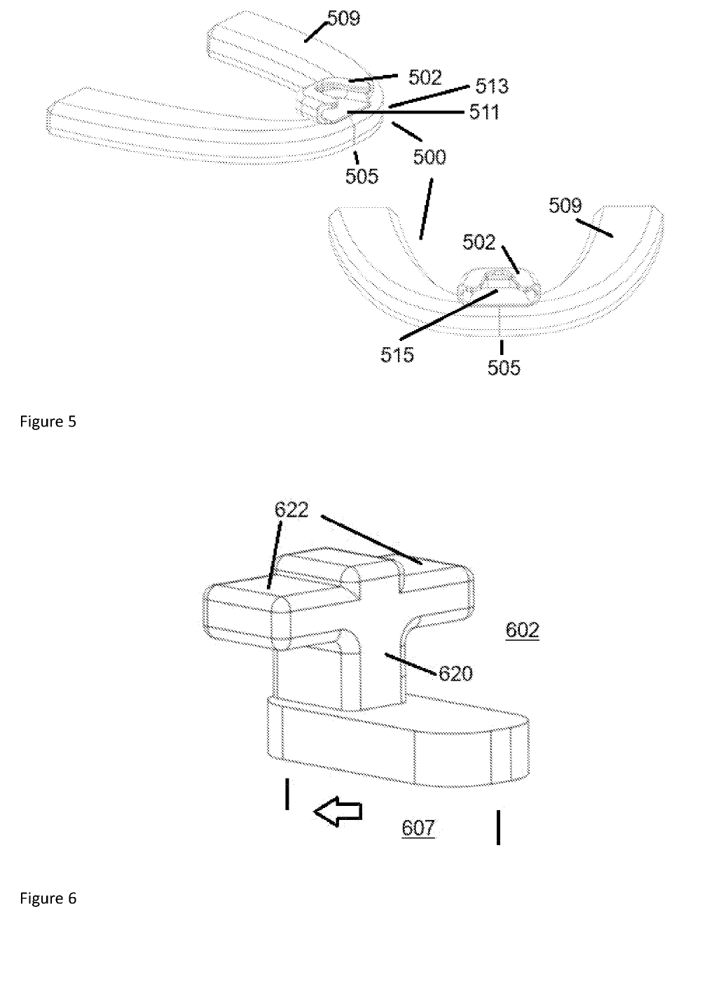

A similar process of incorporating an additional feature is also done with respect to the upper appliance, as is now described. This additional feature is sometimes referred to herein as an upper feature, or upper structural element. In particular, and as shown in FIG. 5 for the first embodiment, the upper feature 502 is added to a front top surface 509 of the upper appliance model 500. Preferably, the position chosen for the additional feature is at the midline 505 of the arch (once again with the midline referenced here with respect to the appliance for convenience of illustration), and with a front portion 511 of the feature 502 approximately aligned with a front 513 of the dental appliance 500. Unlike the lower appliance, however, the feature 502 is fixed and is not offset distally from the incisal edge of the underlying dental arch (in this case the upper). The shape of the upper feature 502 is configured so that the lower feature (302 in FIG. 3, 402 in FIG. 4) can be inserted into the upper feature 502 from a front direction, but otherwise is constrained to remain inside an interior cavity 515 of the upper feature 502.

FIG. 6 shows a more detailed partial view of a configuration of the lower feature 602, also referred to as a lower touch feature. The lower touch feature comprises an upstanding base 620, and a pair of wing elements 622. The lower feature has an offset 607 that is the same offset as shown in FIG. 3.

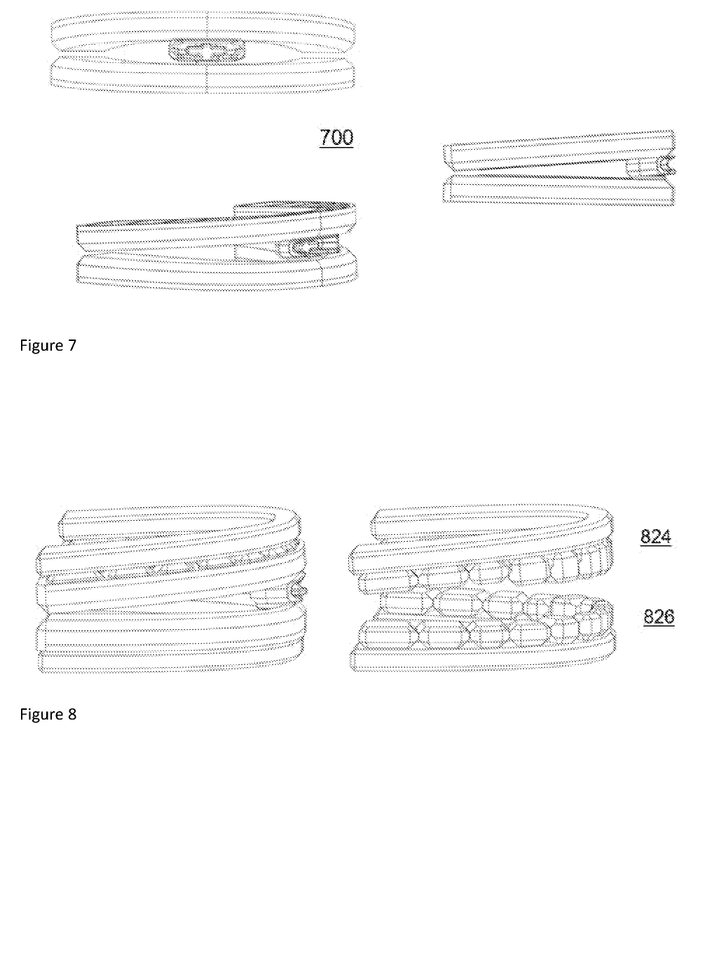

FIG. 7, which shows several views (front, perspective and side) of the mandibular advancement device 700, demonstrates how the lower touch feature fits neatly inside the interior cavity of the upper feature. The only way this lower touch feature can escape the cavity of the upper feature is to move forward (i.e. towards the front of the mouth). The lower appliance is not able to move distally (towards the back of the mouth) because the interior cavity of the upper feature is closed in that direction. Similarly, the lower appliance cannot move vertically since the horizontal bar (in this example the pair of wing elements 622 of the lower touch feature 602 in FIG. 6) prevents the lower feature from exiting the cavity, and a touch point on an upper floor (surface) of the upper feature interior cavity also prevents vertical movement in the other direction. The net result is that there is a single point of contact between the lower appliance and the upper appliance, and that point of contact is only able to move forward or a small distance left and right. The lower appliance is free to rotate as needed because of this single point of contact.

FIG. 8 shows the same configuration as FIG. 7, but with the dental arches 824 and 826 also being depicted. As can be seen when this arrangement is compared to FIG. 1 (the neutral position of the individual’s arches), the positioning of the lower and upper appliances has forced the lower jaw to move forward so that the incisal edges of the lower and upper arches are approximately lined up. The lower jaw is unable to move backward because of the back (distal) wall of the upper feature cavity, but otherwise it is free to rotate or make small movements forwards or sideways. Consequently, the patient using the MAD ensures that the jaws are not “locked” in position or at an unnatural angle, because the single point of contact referenced above allows a comfortable more natural position to be attained.

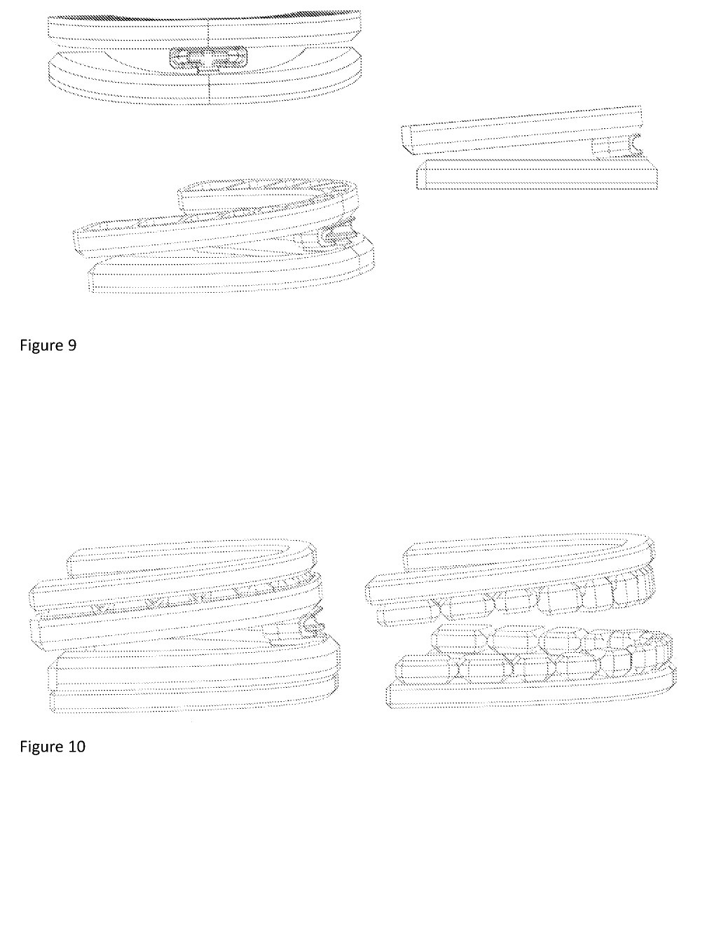

FIG. 9 is similar to FIG. 7 but demonstrates the situation wherein the more distal offset of the lower feature (such as was depicted in FIG. 4) is used. FIG. 10 is similar to FIG. 8 (as the dental arches are also depicted), and shows the effect on the lower jaw relative positioning to the upper jaw. As can be seen, this more distal offset of the lower feature has caused the lower jaw to be forced to move into a more forward position.

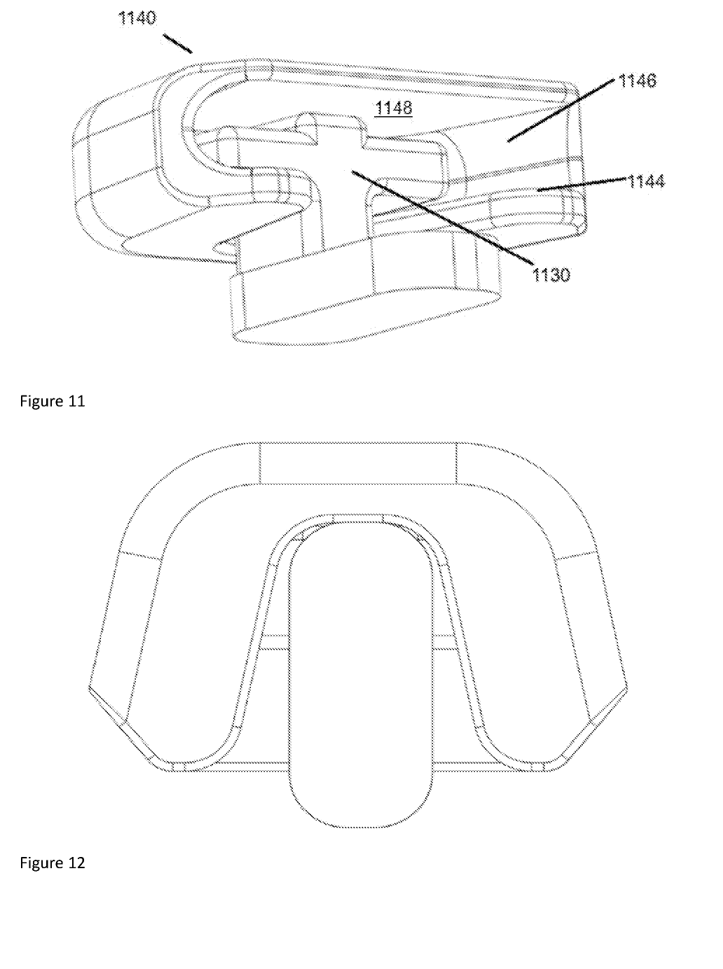

FIG. 11 shows a more detailed view of the lower and upper additional features 1140 and 1130, with the lower feature 1130 being received in the upper feature 1140. In FIG. 11, the interior cavity of the upper feature 1140 is formed by the sidewalls 1146, the lower surface 1144, the upper surface 1148 and a backwall (not seen). The upper feature 1140 has an open front for receiving the lower feature 1130. FIG. 12 is a bottom view of the coupled lower and upper features of FIG. 11 depicting how the lower feature is not necessarily fixed but rather has a small envelope of movement.

Generalizing, and in the manner described, the lower and upper appliances are designed with the additional features to achieve the desired forward movement of the lower jaw and fit benefits.

In general, the medical/dental professionals who are involved with the patient try to find the minimal forward movement of the lower jaw needed to obtain the benefits of the device. This is the process commonly referred to as “titration.” To this end, the following fitting process may be employed. In particular, and for a particular patient, typically there will be a single upper appliance, and a series of lower appliances where the distal offset is progressively increased. In this way, a starting lower offset distance is tried with the patient first. Then, and as needed and according to the feedback of the patient and possible sleep studies, the patient may progress to the next lower appliance in the sequence to test a larger offset. When the correct offset is achieved, the fitting process is terminated but may be revisited in the future.

The following describes a representative step-by-step manufacturing process for the mandibular advancement device of this disclosure in additional detail.

At step (1), 3D models of the lower and upper patient dental arches are obtained. As noted above, these models may be obtained from intraoral 3D scans, or CBCT scans, or from dental impressions or dental models which are then scanned using external 3D digitizers including laser, structured light, moire, or X-ray CBCT devices, or any 3D digitizing device capable of producing a 3D model of the dental arches.

At step (2), a base appliance is generated for each of the lower and upper arches, where the appliance is designed to fit snugly on the patient’s lower and upper dental arches. This operation is typically done on a computer display user interface under the control of a design application (a set of computer program instructions stored in memory or other data store and executed by one or more processors, such as CPUs, GPUs, and combinations thereof). During the design process, the relative mesial/distal movement needed for the lower arch incisal ridge to be made to line up with the upper arch incisal ridge is determined. This is a mesial-distal offset Y.

At step (3), an upper appliance 3D model is created by combining the upper base appliance created in step (2), and combining it with a 3D model of the upper feature cavity. The upper feature cavity is positioned as was shown in the figures.

At step (4), a series of lower appliance 3D models are created by combining the lower base appliance created in step (2), and combining it with a 3D model of the lower touch feature. Each lower appliance shall have a relative offset of ΔY. A value for ΔY of zero shall mean that the lower feature when placed at the back (distal) wall of the upper feature cavity causes the incisal edges of the upper and lower arches to line up. A value for ΔY of 1 shall mean that the lower feature when placed at the back (distal) wall of the upper feature cavity causes the incisal edges of the lower arch to extend a given distance (e.g., 1 mm) mesially (away from the back of the mouth) beyond the incisal edge of the upper arch.

At step (5), and using a 3D printer and an appropriate 3D printing material, the upper appliance is printed together with the sequence of lower appliances to complete the manufacture of the MAD system for the individual patient.

A MAD system comprising the upper and the sequence of lower appliances may be manufactured and delivered with appropriate documentation to a patient as a kit.

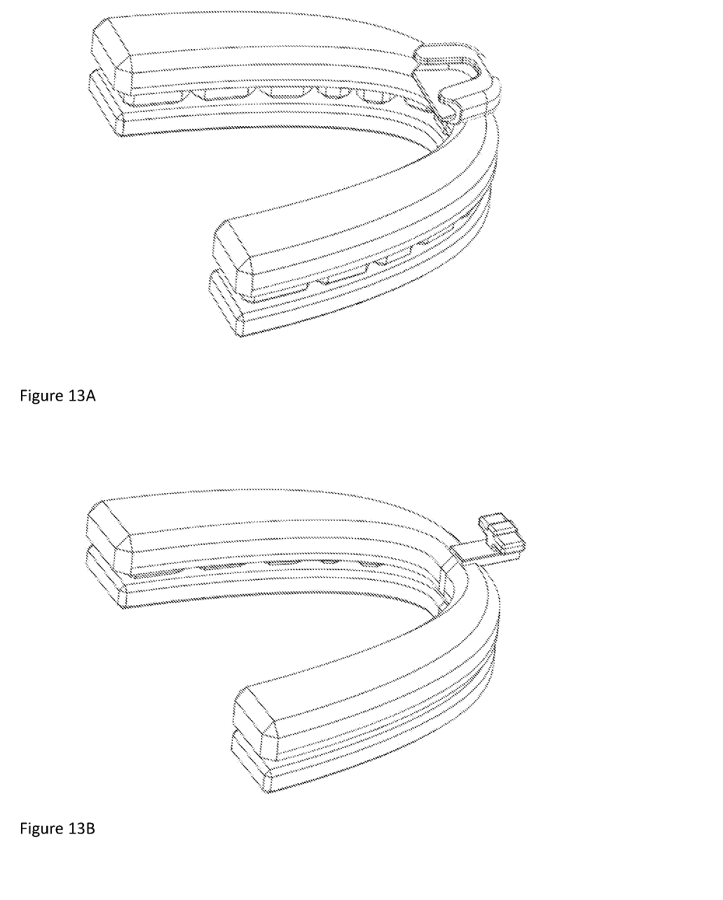

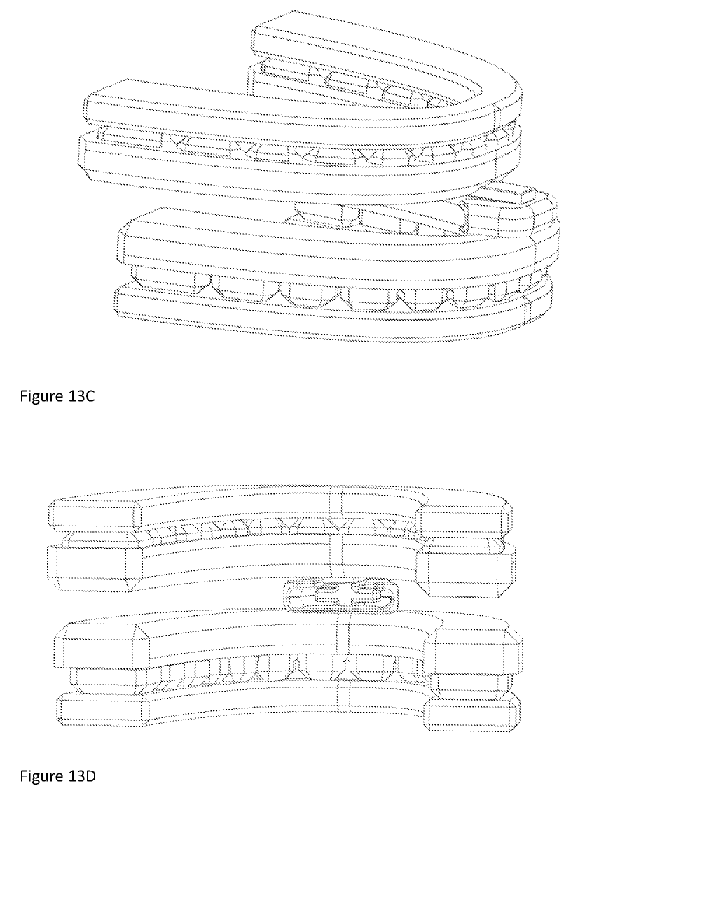

Although the first embodiment as described is preferred, an alternative embodiment may also be implemented wherein the upper and lower features are reversed. This second embodiment is depicted in various views in FIGS. 13A-D. In particular, FIG. 13A depicts the lower appliance positioned on the lower jaw, FIG. 13B is a view from below and that depicts the upper appliance positioned on the upper jaw, FIG. 13C is a perspective view that depicts the appliances coupled together, and FIG. 13D depicts a rear view of the appliances coupled together. The same principles of operation described above apply to this embodiment. That said, in this alternative embodiment the feature that is now on the lower appliance (having been on the upper appliance in the preferred embodiment), has an open cavity facing distally towards the back of the mouth, in contrast to that of the preferred embodiment which has the open cavity facing mesially towards the front of the mouth. This is due to the same requirement in both embodiments of forcing the lower jaw forward. Thus, in this embodiment the second appliance includes a closed cavity having a back-facing opening. The second feature is positioned without the given distal offset and configured to removably receive the first feature through the back-facing opening and to retain the first feature in the closed cavity against forwards and up or down movement when the second appliance is fitted on the patient’s upper dental arch.

### Enabling technologies

3D printing, also called additive manufacturing, is the construction of a three-dimensional object from a CAD model or a digital 3D model. It can be done in a variety of processes in which material is deposited, joined or solidified under computer control, with the material being added together (e.g. plastics, liquids, or powder grains being fused), typically layer by layer.

More specifically, stereolithography is a known technique for making solid objects by successively "printing" thin layers of a curable material, e.g., a radiation-curable material, one on top of the other. To this end, a programmed movable spot beam of light (e.g., UV) shining on a surface or layer of radiation-curable liquid is used to form a solid cross-section of the object at the surface of the liquid. Alternatively, an entire cross section can be cured at once through the use of an LCD or DLP projection technique instead of sweeping a single spot. The object is then moved, in a programmed manner, away from the liquid surface by the thickness of one layer and the next cross-section is then formed and adhered to the immediately preceding layer defining the object. This process is continued until the entire object is formed. Using this printing approach, many different types of object forms can be created using the computer to help generate the programmed commands and to then send the program signals to the stereolithographic object forming subsystem.

Selective Laser Sintering (SLS) using a biocompatible material such as nylon may also be used, in a similar manner to that of stereolithography, except that the source material in this case is a powder, and each layer is cured using heat (for example from a laser), but otherwise the same process is used for building up the appliance through layers. SLS has the added benefit of not requiring support structures, because the appliance continues to be supported by the uncured or unsintered powder.

The particular 3D printer or printing method is not a limitation of this disclosure.

A 3D printer or printing system typically is controlled by an electronic control system. To this end, one or more computers (e.g., servers, network hosts, client computers, integrated circuits, microcontrollers, controllers, field-programmable-gate arrays, personal computers, digital computers, driver circuits, or analog computers) are programmed or specially adapted to perform control tasks, such as: controlling the operation of, or interfacing with, hardware components of the printer system, including a digital micromirror or other optical device (DMD), motors, pumps, valves, and sensors; creating or accessing a digital representation of an object to be fabricated and, based on this digital representation, to control shapes of spatial light patterns (e.g., as projected by the DMD) to fabricate the object by photocuring; controlling motion of a build platform and synchronizing this motion with projected light images, e.g., by controlling timing of steps taken by stepper motors and timing of images projected by a DMD; receiving data from, controlling, or interfacing with one or more sensors; and performing other calculations, computations, programs, algorithms, or computer functions as necessary to facilitate control over the above-described printer or process. One or more thermal cameras may be used for thermal measurement to facilitate software-driven compensation techniques for photopolymerization. In lieu of thermal cameras, a photo-fluorescent dye may be used with a regular camera (e.g., a CMOS-, or CCD-based camera) to enable the control system to quantify how much light is exposed on the photopolymer and then make adjustments with respect to the next layer being printed.

Generalizing, aspects of this disclosure related to the design of the MAD system may be practiced, typically in software, on one or more machines or computing devices.

The computer-assisted design techniques herein may be implemented in a system that is network-accessible and executes as a computing platform.

Each above-described process or process step/operation of the multi-step design process preferably is implemented in computer software as a set of program instructions executable in one or more processors. Such a computer program may be stored in a non-transitory computer readable storage medium, such as, but is not limited to, any type of disk including an optical disk, a CD-ROM, and a magnetic-optical disk, a read-only memory (ROM), a random access memory (RAM), a magnetic or optical card, or any type of media suitable for storing electronic instructions, and each coupled to a computer system bus.

While the above describes a particular order of operations performed by certain embodiments of the invention, it should be understood that such order is exemplary, as alternative embodiments may perform the operations in a different order, combine certain operations, overlap certain operations, or the like. References in the specification to a given embodiment indicate that the embodiment described may include a particular feature, structure, or characteristic, but every embodiment may not necessarily include the particular feature, structure, or characteristic.

While the particular structures of the above-described features have been depicted and described, it should be appreciated that other structural configurations that provide the same functionality may be used. Thus, for example, a lower feature that includes an upstanding structural element of a different shape and that is configured to be inserted into the upper feature from a front direction, but that is otherwise constrained to remain inside an interior cavity, however shaped, of the upper feature, is within the scope of the following claims.

A preferred material of the first and second appliances is Formlabs Dental LT Comfort. More generally, any dental-approved UV-cured biocompatible resin may be used. Preferably, the biocompatible resin is clear.

What is claimed follows below.

1. A dental article of manufacture, comprising:
a first appliance configured to fit dentition of a patient’s lower dental arch and having a first midline, the first appliance comprising a first feature positioned at the first midline; and
a second appliance configured to fit dentition of the patient’s upper dental arch and having a second midline, the second appliance comprising a second feature positioned at the second midline;
the first and second features configured to be coupled together at a single-point-of-contact and at one of: a given distal or mesial offset with respect to an incisal edge of the patient’s dentition.

2. The article of manufacture as described in claim 1,
wherein the first feature is located at the given distal offset with respect to an incisal edge of the patient’s lower dental arch when the first appliance is fitted on the patient’s lower dental arch; and
wherein the second appliance includes a closed cavity having a front-facing opening, the second feature positioned without the given distal offset and configured to removably receive the first feature through the front-facing opening and retain the first feature in the closed cavity against backwards and up or down movement when the second appliance is fitted on the patient’s upper dental arch.

3. The article of manufacture as described in claim 1,
wherein the first feature is located at the given mesial offset with respect to an incisal edge of the patient’s upper dental arch when the first appliance is fitted on the patient’s upper dental arch; and
wherein the second appliance includes a closed cavity having a back-facing opening, the second feature positioned without the given mesial offset and configured to removably receive the first feature through the back-facing opening and retain the first feature in the closed cavity against forwards and up or down movement when the second appliance is fitted on the patient’s lower dental arch.

4. The article of manufacture as described in claim 2, wherein the first feature comprises an upstanding structure, and a lateral structure supported on the upstanding structure.

5. The article of manufacture as described in claim 1, wherein the given distal or mesial offset is configured to be approximately equal to a desired distance of lower jaw movement.

6. The article of manufacture as described in claim 1, wherein the first and second appliances are formed of a dental-approved UV-cured clear biocompatible resin.

7. The article of manufacture as described in claim 1, wherein the first and second appliances are formed of a dental-approved Selective Laser Sintering (SLS) material.

8. The article of manufacture as described in claim 1, wherein the first appliance is one of a set of first appliances each with a different given distal offset.

9. The article of manufacture as described in claim 1, wherein the second appliance is one of a set of second appliances each with a different given mesial offset.
